(19) United States
(12) Patent Application Publication
(10) Pub. No.: US 2019/0216333 A1
(43) Pub. Date: Jul. 18, 2019

(54) THERMAL FACE IMAGE USE FOR HEALTH ESTIMATION

(71) Applicant: Futurewei Technologies, Inc., Plano, TX (US)

(72) Inventors: Jui-Hsin Lai, San Jose, CA (US); Yinglong Xia, San Jose, CA (US); Jen-Hao Hsiao, Santa Clara, CA (US); Yu Zhang, Beijing (CN)

(21) Appl. No.: 15/870,617

(22) Filed: Jan. 12, 2018

(51) Int. Cl. A61B 5/01; G16H 50/20; A61B 5/00; G06F 17/30

(52) U.S. Cl. CPC A61B 5/01; G16H 50/20; A61B 5/0022; G16H 50/30; A61B 5/742; A61B 5/0064; G06T 2207/10024; G06F 17/30247

(57) ABSTRACT

A computer implemented method includes capturing, via a camera, one or more digital images of a face of a person representative of blood circulation of the person, collecting context information via one or more processors corresponding to the person contemporaneously with the capturing of the one or more digital images, labeling, via a trained individual health model executing on the one or more processors, the one or more digital images based on the blood circulation represented in the image and the collected context information via the trained individual health model that has been trained on prior such digital images and context information; and analyzing, via the one or more processors, the one or more labeled digital images to generate a health index of the person.

### TECHNICAL FIELD

The present disclosure is related to systems that calculate health indices, and in particular to a system and method that utilizes thermal information in facial images to provide an estimation of a person's health.

### BACKGROUND

Personalized in-home family care has many advantages. The use of applications executing on smart devices can make it very convenient for users to monitor and evaluate their health on a daily basis without going to a hospital. Most vital signs can be taken at home, which may be a single dwelling, apartment, condominium, assisted living facility, or other place where a person lives. It's much more pleasant to take vital signs, such as temperature, blood pressure, glucose, etc., at home instead of in a hospital. The application to assist with such monitoring can be beneficial for people who have little access to medical care, like those living in suburbs, small towns, or remote regions in short supply of medical care facilities.

With smartphones and wearable devices becoming ubiquitous, mobile healthcare (mHealth) is getting popular. Mobile applications (APPs) have been proposed to calculate a personal health index, and some APPs may even provide for disease management.

### SUMMARY

Various examples are now described to introduce a selection of concepts in a simplified form that are further described below in the detailed description. The Summary is not intended to identify key or essential features of the claimed subject matter, nor is it intended to be used to limit the scope of the claimed subject matter.

According to one aspect of the present disclosure, a computer implemented method includes capturing, via a camera, one or more digital images of a face of a person representative of blood circulation of the person. Context information is collected via one or more processors. The context information corresponds to the person, and is collected contemporaneously with the capturing of the one or more digital images. A trained individual health model executing on the one or more processors is used to label the one or more digital images based on the blood circulation represented in the image and the collected context information. The trained individual health model has been trained on prior such digital images and context information. The one or more processors are used to analyze the one or more labeled digital images to generate a health index of the person.

Optionally, in any of the preceding aspects, a further implementation of the aspect includes wherein the one or more digital images comprise infrared (IR) images.

Optionally, in any of the preceding aspects, a further implementation of the aspect includes wherein the one or more digital images comprise RGB (red, green, blue) images.

Optionally, in any of the preceding aspects, a further implementation of the aspect includes wherein the one or more digital images are captured at a same time each day comprising a time proximate a waking or going to sleep time and the context information is collected contemporaneously with the capture of the one or more digital images.

Optionally, in any of the preceding aspects, a further implementation of the aspect includes wherein collecting context information comprises collecting input by the person regarding how the person is feeling.

Optionally, in any of the preceding aspects, a further implementation of the aspect includes wherein the individual health model comprises a convolutional neural network (CNN) trained with labeled images of the person, wherein the labels comprise medical conditions.

Optionally, in any of the preceding aspects, a further implementation of the aspect includes providing the labeled digital images and context information from the individual health model to a general health model, and receiving health condition information from the general health model responsive to the provided labeled digital images and context information, and using such received health condition information from the general health model in generating the health index.

According to one aspect of the present disclosure, a device includes a memory storage comprising instructions, a camera, and one or more processors in communication with the memory storage and camera. The one or more processors execute the instructions to capture, via the camera, one or more digital images of a face of a person representative of blood circulation of the person, collect context information corresponding to the person contemporaneously with the capturing of the one or more digital images, label, via a trained individual health model executing on the one or more processors, the one or more digital images based on the blood circulation represented in the digital images and the collected context information via the trained individual heal model that has been trained on prior such digital images and context information, and analyze the one or more labeled digital images to generate a health index representative of the health of the person.

Optionally, in any of the preceding aspects, a further implementation of the aspect includes wherein the one or more digital images comprise infrared (IR) images.

Optionally, in any of the preceding aspects, a further implementation of the aspect includes wherein the one or more digital images are captured at a same time each day comprising a time proximate a waking or going to sleep time and the context information is collected contemporaneously with the capture of the one or more digital images.

Optionally, in any of the preceding aspects, a further implementation of the aspect includes wherein the individual health model comprises a convolutional neural network (CNN) trained with labeled images of the person, wherein the labels comprise medical conditions.

Optionally, in any of the preceding aspects, a further implementation of the aspect includes wherein the one or more processors execute instructions to provide the labeled digital images and context information from the individual health model to a general health model, and receive health condition information from the general health model responsive to the provided labeled digital images and context information, and use such received health condition information from the general health model in generating the health index.

Optionally, in any of the preceding aspects, a further implementation of the aspect includes wherein the one or more processors execute instructions to generate a notification including health advice for the person.

Optionally, in any of the preceding aspects, a further implementation of the aspect includes wherein the device comprises a cellular phone with an integrated camera having a microbolometer array for capturing the digital images of the person representative of blood circulation.

According to one aspect of the present disclosure, a non-transitory computer-readable media storing computer instructions for generating a health indication, that when such computer instructions are executed by one or more processors cause the one or more processors to perform operations. The operations include capturing, via a camera, one or more digital images of a face of a person representative of blood circulation of the person, collecting context information via one or more processors corresponding to the person contemporaneously with the capturing of the one or more digital images, labeling, via a trained individual health model executing on the one or more processors, the one or more digital images based on the blood circulation represented in the digital images and the collected context information via the trained individual heal model that has been trained on prior such digital images and context information, and analyzing, via the one or more processors, the one or more labeled digital images to generate a health index representative of the health of the person.

Optionally, in any of the preceding aspects, a further implementation of the aspect includes wherein the individual health model comprises a convolutional neural network (CNN) trained with labeled images of the person, wherein the labels comprise medical conditions, and wherein the labeled and captured images comprise infrared (IR) images.

Optionally, in any of the preceding aspects, a further implementation of the aspect includes wherein executing the instructions further causes the one or more processors to perform operations including providing the labeled digital images and context information from the individual health model to a general health model, and receiving health condition information from the general health model responsive to the provided labeled digital images and context information, and using such received health condition information from the general health model in generating the health index.

### BRIEF DESCRIPTION OF THE DRAWINGS

FIG. 1 is a processing flow diagram of a system for generating a health index based at least in part on an image of a person that represents the person's thermal condition according to an example embodiment.

FIG. 2 is a flowchart illustrating a method of generating a health index from thermal face images and context information according to an example embodiment.

FIG. 3A is a representation of a thermal face image that illustrates a region of interest according to an example embodiment.

FIG. 3B is a block diagram illustrating classification of an image and associated context information by a trained convolutional neural network (CNN) according to an example embodiment.

FIG. 4 is a representation of an algorithm pipeline for a deep learning network used for training the individual health model according to an example embodiment.

FIG. 5 shows three different thermal images of the same person with different labels according to an example embodiment.

FIG. 6 is a representation of example context information shown on a display screen according to an example embodiment.

FIG. 7 is a representation of further context information relating to weather associated with a person according to an example embodiment.

FIG. 8 is a collection of multiple thermal face images from multiple different people that may be provided to a general health model according to an example embodiment.

FIG. 9 is a representation of a screen shot illustrating an appointment booking interface according to an example embodiment.

FIG. 10 is a block diagram illustrating circuitry for clients, servers, cloud based resources for implementing algorithms and performing methods according to example embodiments.

### DETAILED DESCRIPTION

In the following description, reference is made to the accompanying drawings that form a part hereof, and in which are shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software in one embodiment. The software may consist of computer executable instructions stored on computer readable media or computer readable storage device such as one or more non-transitory memories or other type of hardware based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine.

The human body is complicated system and people have their own living habits and different environment contexts, which makes the prediction of health difficult for APPs. There is a need to train a customized health model for everyone, but the challenge is how to collect an individual's training data for customized healthcare analysis.

It is difficult to find a general health model the works effectively for all people. Collecting data for each individual person from which a representation of the health of an individual can be assessed or predicted can be difficult. Such collected data has generally lacked sufficient information to provide a reliable assessment or prediction. Data collected has relied on devices to collect vital signs. While vital signs can be helpful in determining the health of a person, the vital signs may lack accuracy to make such a determination and do not provide sufficient data to predict the health of the person in the future.

In various embodiments, a smart device executing a health care related application or applications may be used to facilitate monitoring of the health condition of a person. Data collected by the smart device may include images, such as face images, of the person containing thermal information representative of blood circulation. Such images may be infrared (IR) based images or red-green-blue (RGB) images from which the thermal information may be extracted. Other information, such as vital signs may also be received or detected by the smart device or from external devices, such as wearable devices. Still further, the smart device may detect or receive context information about the person/user, such as body type, previously diagnosed conditions, sound, calendar events, wearable device inputs, weather related data, pollen counts, pollution levels, activity levels, time of day, etc. Many different kinds of vital sign data can be collected via a smartphone and wearable devices to provide informative cues in healthcare analysis or disease detection.

Information of physical activity from wearable devices has its limitation in disease analysis. RGB sensors on smartphones also have some limitations in detecting symptoms under the skin. In various aspects of the present inventive subject matter, a smart device or other system collects and trains a customized health model for health index estimation by analyzing thermal face images. The analyzed thermal face images may be used in conjunction with the context information to provide an estimate, such as a health index, of a person's health.

Such context information and images may be processed by the smart device to monitor the health of the person. The smart device may also or alternatively be used by an in-home health care provider, including a family member, or friend for example to use the context information and images of the person to generate a personalized health rating. The smart device, either alone or in combination with network or cloud based resources, may label and train individual health models for the person. Multiple smart devices used by multiple people may provide labeled images and context information to network based general health models that can provide epidemic disease detection.

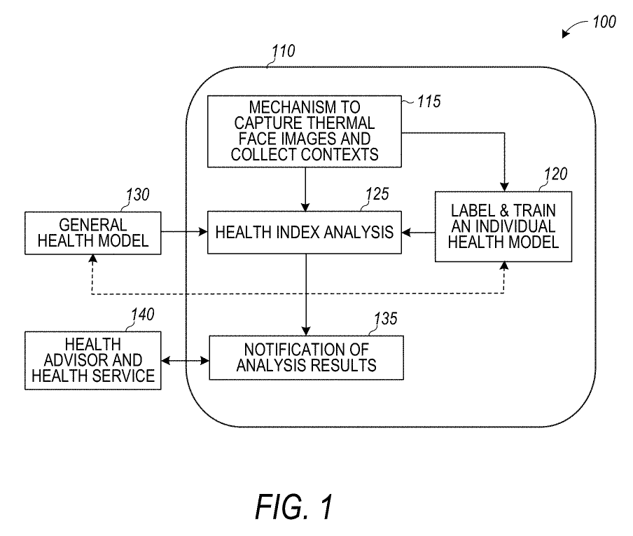

FIG. 1 is a processing flow diagram of a system 100 for generating a health index based at least in part on an image of a person that represents the person's thermal condition. The thermal condition may be indicative of blood circulation, referred to as a person's circulatory condition. The system 100 may include a smart device 110, such as a smart phone, touchpad, robot, or other device which may be a mobile device or stationary device such as a personal computer or other computer system.

The smart device 110 includes a mechanism 115 to capture a person's profile images. The mechanism 115 may be a camera which includes at least one of RGB and thermal sensors, such as an array of microbolometers to capture infrared radiation from the person. Both types of images, RGB and thermal sensor based images, may be used to detect the status of a person's circulatory condition. The camera may be front facing or rear facing and integrated into the smart device 110. A front facing camera enables easier use by the person to capture their own image, while a rear facing camera enables health care providers, either professional, friends, or family to capture the person's image.

Both types of images, RGB and thermal sensor images, may be used to detect the status of a person's circulatory condition. For example, a higher temperature shown in the image may indicate denser capillaries and better blood circulation. Temperature distribution and changes may map to the status of the circulatory system, with different distributions being associated with different health conditions. Thermal sensor based images contain robust pixel information that clearly reflects such temperatures and temperature distributions. While RGB based images contain thermal information, it may be more difficult to extract temperature distributions, and hence circulatory conditions as compared to infrared based images. RGB images may also be biased by cosmetics and background lighting.

Taking of images by the smart device 110 may be triggered when the person wakes up, such as when turning off an alarm on the smart device 110 or browsing APPs in the morning shortly after waking up. An alarm to take the image or images at the same time each day, such as waking or going to sleep, may also be used. The alarm may continue until the person takes a picture of their face that is sufficient for capturing the thermal/circulatory related information. Capture of an image or images may also be triggered at bed time, such as when a user sets up an alarm or uses the smart device when laying down at night. Since thermal imaging of the face may be biased by activity, taking the images at a set time when the person is less likely to be as active as during the day, helps remove such activity based biasing.

The mechanism 115 may also be used to capture or collect context information about the person, such as weather conditions, room temperature, and activity information. Room temperature may be derived from background thermal image data, while activity may be inferred from heart rate information collected from a wearable device or even input by a person or other device/sensor. A wearable device may also track steps and provide information about when the person was walking, running, climbing, or sedentary.

The smart device 110 may also include a training module 120 that may include circuitry and instructions for labeling the collected images. Labeling of the images may be performed based on information provided by the person or healthcare provider, such as via a popup window with the ability to input information about how the user is feeling, or to directly label an image from a dropdown menu of predefined labels such as headache, runny nose, sore throat, normal, tired, etc.

Labeling may further be performed as a function of sound captured by a microphone integrated into or communicatively coupled to the smart device 110, wearable device inputs, and calendar events. For instance, if a person is exercising, the training module 120 may label the image with an indication that the image reflects a context of exercising or physical exertion. The microphone may pick up sounds indicative of coughing or sneezing, sniffling, running nose, nose blowing, moans for pain, or other sounds that may be correlated to certain health conditions, and label the collected images accordingly. Such sounds may be picked up during a phone call made using the smart device 110, or even contemporaneously with collection of the images. In one embodiment, the training module 120 may use a convolutional neural network (CNN) to train an individual health model using the collected thermal images and corresponding labels as described in further detail below.

The individual health model, which is also represented in FIG. 1 by training module 120, when trained, provides predictions of the thermal condition reflected in the images to a health index analysis module 125, which also receives context information collected by the mechanism 115. The health index module 125 analyzes the thermal condition information and context information using statistically sound methods, which may weight various pieces of information to generate a health index.

In one embodiment, the health index may be calculated in the following manner:

Overall health index:

S=w_1F1(x_1,x_2, . . . ,x_n)+w_2F2(x_1,x_2, . . . x_n)+ . . . +w_k Fn(x_1,x_2, . . . ,x_n)

An individual health score, Si, for the i-th disease (e.g., the higher the score, the lower probability to have the i-th disease at the moment):

Si=Fi(x_1,x_2, . . . ,x_n)

where S is the overall health index or score; x_1, x_2, . . . , x_n are the parameters (For example, if the parameters, x, used are blood pressure, body weight, and thermal image to calculate the health index, n=3.); and Si=Fi(x_1, x_2, . . . , x_n) is a predictor for predicting the risk of a specific disease (e.g., fever, anemia, . . . ) based on parameters x, and can be learned automatically from machine learning algorithms, or set heuristically based on expert experience (e.g., a rule-based method). w_1, w_2 . . . w_k are the weights associated with each disease predictor to give an overall health score. The weights can be set heuristically based on expert experience, be learned automatically from machine learning algorithms, or simply a uniform distribution among all individual health scores.

The health index or score may be a number representative of the overall health of the person. The rating may be scaled in some embodiments to a score of zero or 1 to 100, with either zero or 1, or 100 being indicative of good or bad health in various embodiments. Other ranges may be used for the scale, such as 0-10 for example.

The health index may be provided to a notification module 135, which provides notifications to the person. Example notifications based on the health index may include an indication that the person appears to be coming down with a cold or the flu, and to rest, drink plenty of liquids, and try to consume certain types of nutrition. If the person's temperature is high for example, the notification may also indicate that the person should seek a health advisor indicated by the health service 140, and may even notify the health advisor or service.

The notification module 135 may also include or have access to a cloud based health database which may be accessed by use of the health index to retrieve information for provision to the person regarding health care recommendations. In some embodiments, the health service 140 may provide appointment making capabilities, and respond with available appointments and facilitate arranging an appointment to be placed on the person's calendar on the smart device. For example, if the person's temperature is higher than a threshold, such as 103 F, an appointment schedule may be displayed on the smart device 110. The threshold may also be dependent on a person's profile, and may be lower for transplant recipients or a person with a history of certain types of illnesses. The contact information for the health advisor or service may be included in the person's profile, which may be stored in memory of the smart device 110.

As described above and shown in FIG. 1, the smart device 110 may include the mechanism 115, the training module 120, the health index analysis module 125, and the notification module 135, which may be integrated into the smart device 110. The smart device may also include a display screen, such as a touch screen for display and input by the person or other user providing services to the person.

In some embodiments, the images and labels may also be provided to a general health model 130, which may be executing on cloud based resources. The general health model 130 may receive labeled images from multiple different persons, such as thousands of persons. In some embodiments, some contact information may also be included, such as physical locations of the persons. The general health model 130 may utilize the same deep learning type of network, and CNN used for the individual health model in training module 120 in some embodiments. However, with data from many different people, the general health model may be able to spot health trends, such as epidemics or other public health issues and create corresponding epidemic disease models. The general health model 130 may also be able to provide indications of likely causes for health conditions based on previous similar cases as reflected in the thermal face images received from many other people. Such indications may be provided to the health index analysis module 125, which may be able to provide a more informative notification, using the notification module 135, to the person and health advisor or health service 140.

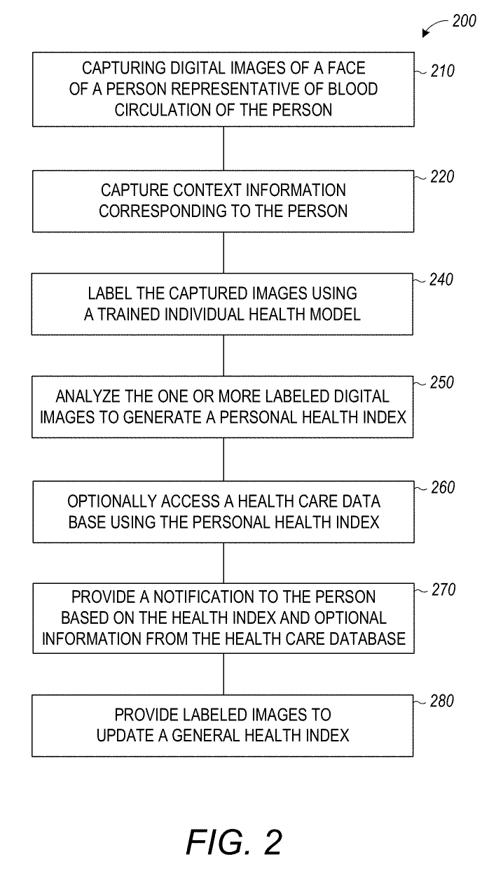

FIG. 2 is a flowchart illustrating a method 200 of generating a health index from thermal face images and context information. The method 200 begins by capturing at operations 210, via a camera, one or more digital images of a face of a person representative of blood circulation of the person. At operations 220, context information is captured corresponding to the person contemporaneously with the capturing of the one or more digital images. Capturing context information contemporaneously involves capturing context information at a time about the capturing of the image such that the context information captured is relevant to the thermal information contained in the image. Some context information may be captured within seconds or minutes before or after capture of the image, like activity information and current room temperature, and input from the person such as a label or other indication of how the person is feeling at the time. Such labels are indications of medical conditions, and may include how the person is feeling or actual diagnoses, such as sore throat, headache, normal, etc. Other context information which may not change fast, such as user profile information, may be captured within an hour or longer before or after the image.

Labeling of the captured image or images, at operations 240, is performed by a trained individual health model such that labels of the one or more digital images are based on the blood circulation represented in the image and the collected context information. The individual health model may be a deep learning network such as a CNN trained on prior such images and context information. Operations 250 analyze the one or more labeled digital images to generate a health index for the person.

At operations 260, the method 200 may optionally access a health care database using the personal health index. The health care database may provide information regarding potential conditions based on the index, such as care recommendations including nutritional guidance and may also recommend an appointment with health care provider.

Operations 270 may be used to provide a notification to the person based on the health index and optional information from the health care database. Operations 280 may be executed to provide labeled images to update a general health index. The general health index may also provide information back to operations 250 for use in generating the health index.

In some embodiments, the image is an RGB digital image or may be a microbolometer/IR based image. The images may be collected at times of known activity, such as near waking time or bedtime, when activity levels are lowest and least likely to mask thermal image information representative of the health of the person.

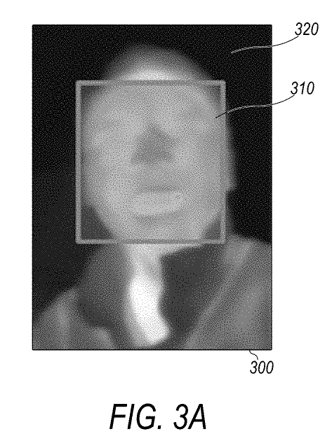

FIG. 3A is a representation of a thermal face image 300 that illustrates a region of interest 310 and a background 320. Note that the image is reproduced herein as a black and white image, but the different shades of black still show different temperatures of the face. Color images show temperature variations as different colors and intensities of color, however, the black and white image still conveys that there are color differences. The pixel data behind the images is what is processed to create the individual health model. The region of interest 310 is detected and may include various sub-regions of interest such as eyes, mouth, nose, forehead, etc. Various weighting for the different sub-regions may be used during training of the individual health model by training module 120 and determined via the training using a CNN. Note also that context information may be obtained from image 300. The background 320 color is representative of a temperature of the environment in which the image 300 was taken. Thus, the temperature of the environment, most likely a room in a dwelling may be derived from the background pixel information of the image 300. The derived temperature may be added to the context information provided for labeling and training the individual health model by training module 120.

FIG. 3B is a high-level block diagram illustrating classification of image 300 and associated context information 330 that is provided to a trained convolutional neural network (CNN) 340. The pixel information and context information are provided in digital form to the trained CNN 340, which generates an output 350, which corresponds to a classification of the information fed to the CNN 340. Essentially, the classification is a label that the CNN 340 learned from the labeled training data used to train the CNN 340. Example labels are shown as headache, anemia, flu, fever, etc. Note that the CNN 340 may be able to classify pictures of users for many more different maladies that are not perceivable by humans. The output is then used to generate a health index 360 for the user.

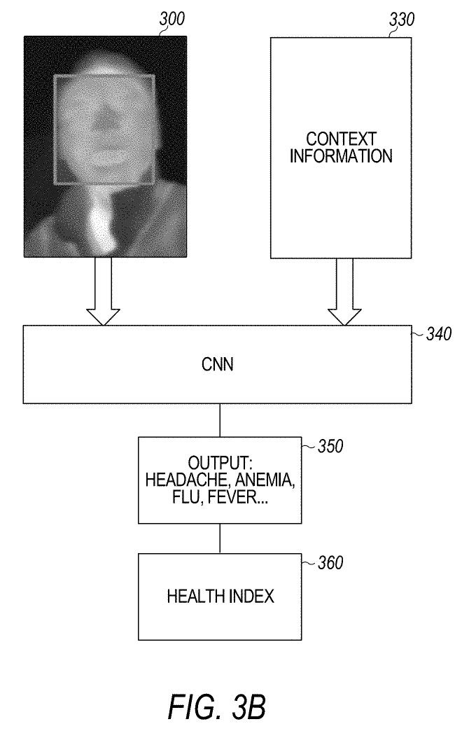

FIG. 4 is a representation of an algorithm pipeline for a deep learning network 400 used for training the individual health model using a CNN. The network 400, in one embodiment, is a 3-layer CNN. The term 3-layer corresponds to the use of three convolutional layers in the network. Convolutional layers identify features at different levels of abstraction, with the first layers looking for such things a straight lines, curved lines, edges, temperatures, etc., while later layers identify higher level features that are more likely to be directly associated with identification of the subject, or in this case, health condition associated with the person in the image. While a CNN is shown for illustrative purposes, other types of deep learning networks may be used in further embodiments, such as ResNet (residual network), Inception, Exception, VGG 16, and others.

In one embodiment, images and associated context 410 are provided to the network 400 for either training or labeling. The context information, such as activity level for example, is encoded for use by the network 400. In one example, the activity level may be sensed by an accelerometer of the smart device 110 and encoded as a digital representation of a number of activity levels from sedentary to extremely active. During the training stage, the network is being trained, and images may be provided with labels as part of the context information. Such provided labels may be specified by the person as previously described. Note that training may also continue during normal use when the network is used to determine the labels for the images.

A first convolutional layer 410 provides an output that comprise first feature maps 420. The first feature maps 420 are maps of pixels and their intensity and temperature or color depending on the type of image, as well as the bits representing the associated context. Each bit is provided to a respective neuron of the first convolutional layer 410. A subsampling layer 425 normalizes the first feature maps 420 and partitions the input image into a set of non-overlapping rectangles and for each such sub-region, outputs a second feature map 430.

The second feature map 430 is provided to a second convolutional layer 435 that produces third feature maps 440 that are subsampled at subsampling layer 445 to produce fourth feature maps 450 that are provided to a fully connected layer 455.

The fully connected layer 455 looks at the output, fourth feature maps 450, and determines which features most correlate to a label representative of a particular class. Basically, the fully connected layer 455 determines what high level features most strongly correlate to a particular class and has particular weights such that computing products between the weights and the previous layer, correct probabilities for the different classes are obtained as indicated at output 460. Note that in this case, the classes correspond to health condition related labels corresponding to the thermal images of the person. Example labels shown include headache, anemia, flu, fever, etc.

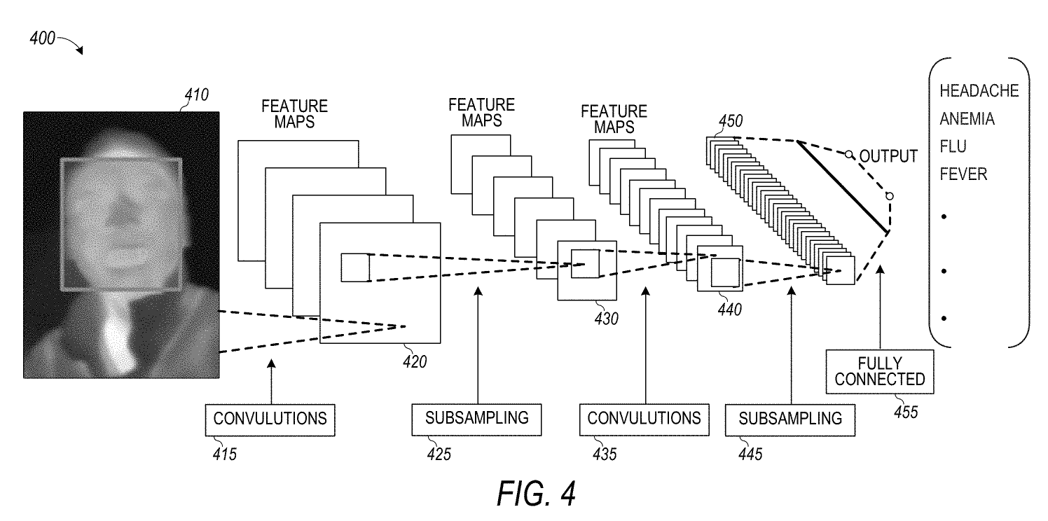

FIG. 5 shows three different thermal images of the same person with different labels. Image 510 shows a heat distribution that represents a person with normal health. Image 520 shows a heat distribution for the same person that represents a running nose. Image 530 shows a heat distribution for the same person who now has a headache. The network 400 has been trained as described above to recognize these images and apply the corresponding label to them. The same images may also be pre-labeled and used during the training stage, or further used to continue training the network 400 during operation.

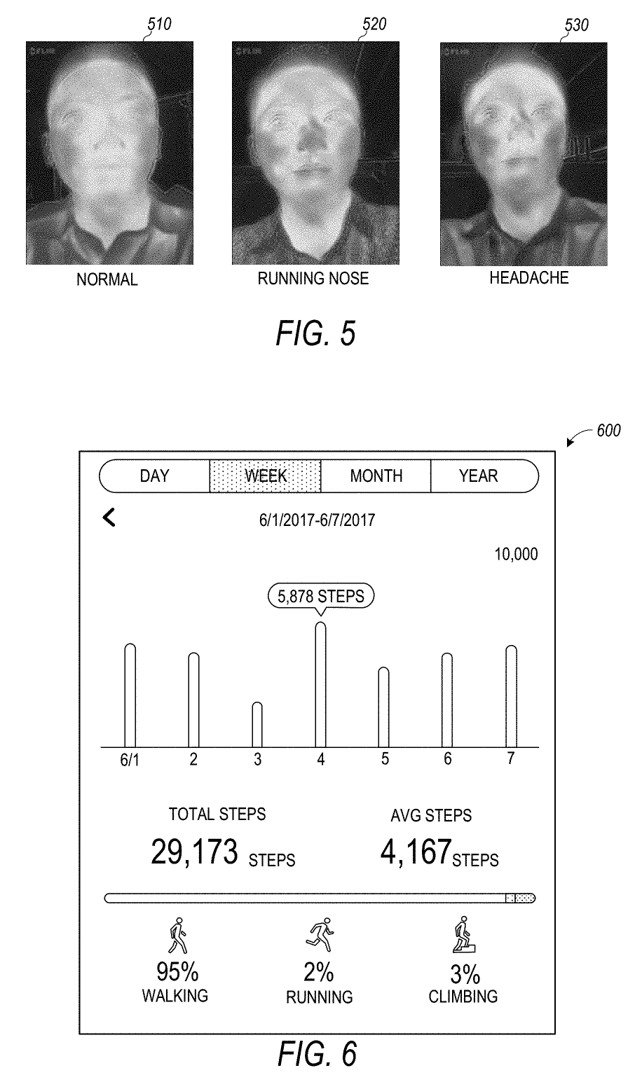

FIG. 6 is a representation of example context information shown on a display screen of the smart device at 600. This context information may be obtained from a wearable device that counts steps and can also determine whether the wearer is running, walking, or climbing. Thus the wearable device may include various accelerometers, timers, altimeters, and other sensing devices, such as pulse rate sensors, temperature sensors, etc. The data displayed is displayable by day, week, month, and year. Some or all of the data developed by the wearable device may be provided to the smart device via a short distance wireless protocol, such as a Bluetooth® protocol. In other words the wearable device may be paired with the smart device. Note that selected data may form part of the context associated with a face image obtained by the smart device.

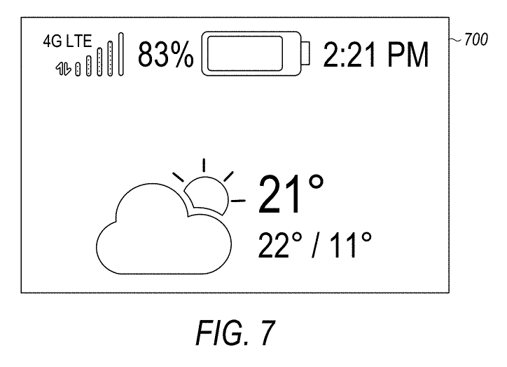

FIG. 7 is a representation of further context information at 700 relating to weather associated with a person using the smart device. Such context information 700 may also be used by training module 120 to train the individual health model. Information 700 may be obtained via a weather app running on the smart device in some embodiments, and may include for example, temperature, sun and cloud conditions, moon phase, humidity, pollen counts, UV indices, pollution indications, and any other information which may be relevant in calculating a health index for the person.

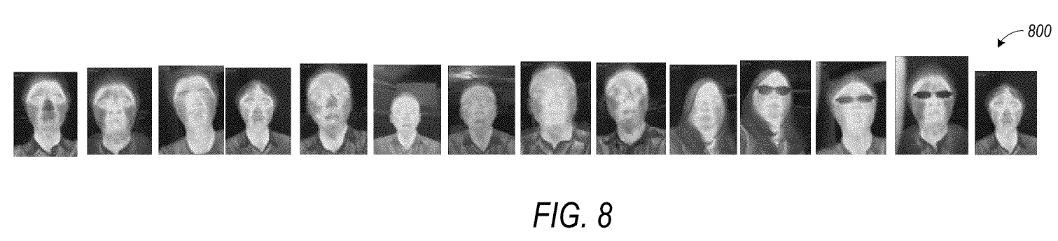

FIG. 8 is a collection 800 of multiple thermal face images from multiple different people that may be provided to the general health model 130 from various smart devices used by the different people. Each of the images in the collection 800 may include a label derived from the respective smart devices as well as context information for use by the general health model 130 in training the general health model and in providing information back to each smart device health analysis module 125 in determining the personalized health index.

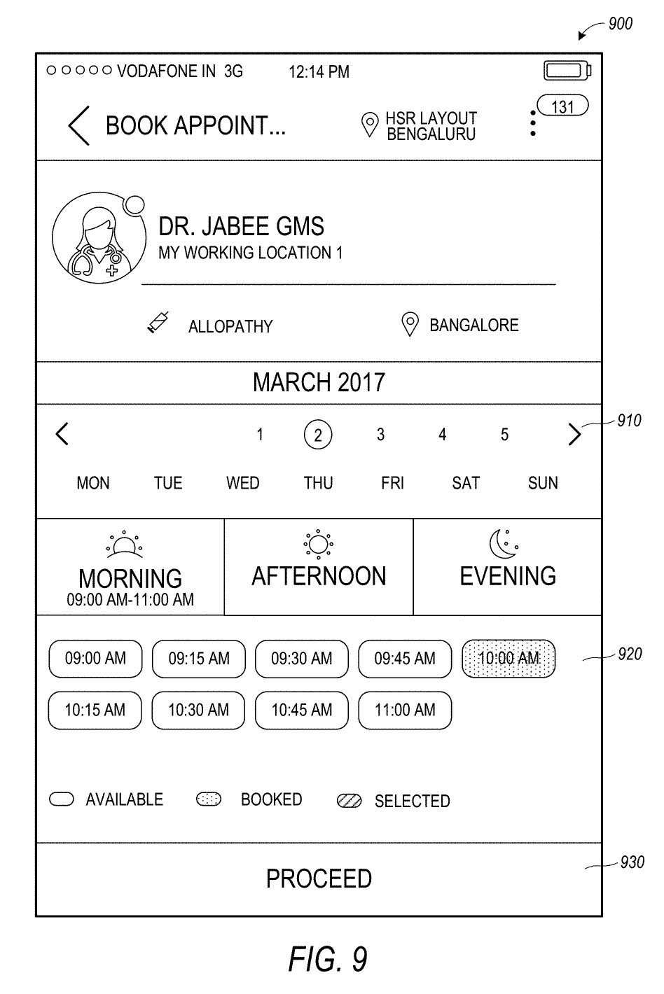

FIG. 9 is a representation of a screen shot 900 illustrating an appointment booking interface. The screen shot 900 may be shown on the display of the smart device 110 via interaction with the health service 140. An appointment app on the smart device 110 may interface with the health service 140 to generate the screen shot 900 and illustrate a name of a health advisor, shown as “Dr. jabee gms”, and dates 910 and times 920 that are available. A user of the smart device 110, such as the person whose health is being analyzed may use a touch screen or other user interface to select the dates and times and also select a button 930 to proceed to make the appointment. Screen 900 may be generated automatically as a result of the notification module 135 providing results to the health advisor and the health service 140. Such results may include the health index and/or additional context data, and even the digital images. The results may also include an urgency of the person being seen by the health professional, and may result in a recommendation for the person to go to an emergency room rather than wait for an available appointment. Such a recommendation may be generated by the health service 140 or by the notification module 135.

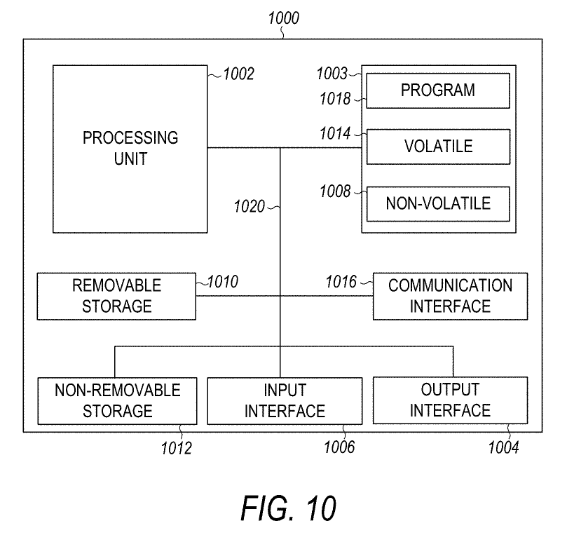

FIG. 10 is a block diagram illustrating circuitry for estimating health of a person using thermal face images and context information and performing methods according to example embodiments. All components need not be used in various embodiments.

One example computing device in the form of a computer 1000 may include a processing unit 1002, memory 1003, removable storage 1010, and non-removable storage 1012. Although the example computing device is illustrated and described as computer 1000, the computing device may be in different forms in different embodiments. For example, the computing device may instead be a smartphone, a tablet, smartwatch, or other computing device including the same or similar elements as illustrated and described with regard to FIG. 10. Devices, such as smartphones, tablets, and smartwatches, are generally collectively referred to as mobile devices or user equipment. Further, although the various data storage elements are illustrated as part of the computer 1000, the storage may also or alternatively include cloud-based storage accessible via a network, such as the Internet or server based storage.

Memory 1003 may include volatile memory 1014 and non-volatile memory 1008. Computer 1000 may include—or have access to a computing environment that includes—a variety of computer-readable media, such as volatile memory 1014 and non-volatile memory 1008, removable storage 1010 and non-removable storage 1012. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 1000 may include or have access to a computing environment that includes input interface 1006, output interface 1004, and a communication interface 1016. Output interface 1004 may include a display device, such as a touchscreen, that also may serve as an input device. The input interface 1006 may include one or more of a touchscreen, touchpad, mouse, keyboard, camera, one or more device-specific buttons, one or more sensors integrated within or coupled via wired or wireless data connections to the computer 1000, and other input devices. The computer may operate in a networked environment using a communication connection to connect to one or more remote computers, such as database servers. The remote computer may include a personal computer (PC), server, router, network PC, a peer device or other common network switch, or the like. The communication connection may include a Local Area Network (LAN), a Wide Area Network (WAN), cellular, WiFi, Bluetooth, or other networks. According to one embodiment, the various components of computer 1000 are connected with a system bus 1020.

Computer-readable instructions stored on a computer-readable medium are executable by the processing unit 1002 of the computer 1000, such as a program 1018. The program 1018 in some embodiments comprises software that, when executed by the processing unit 1002, performs network switch operations according to any of the embodiments included herein. A hard drive, CD-ROM, and RAM are some examples of articles including a non-transitory computer-readable medium such as a storage device. The terms computer-readable medium and storage device do not include carrier waves to the extent carrier waves are deemed too transitory. Storage can also include networked storage, such as a storage area network (SAN). Computer program 1018 may be used to cause processing unit 1002 to perform one or more methods or algorithms described herein.

### EXAMPLES

In example 1, a computer implemented method includes capturing, via a camera, one or more digital images of a face of a person representative of blood circulation of the person. Context information is collected via one or more processors. The context information corresponds to the person, and is collected contemporaneously with the capturing of the one or more digital images. A trained individual health model executing on the one or more processors is used to label the one or more digital images based on the blood circulation represented in the image and the collected context information. The trained individual health model has been trained on prior such digital images and context information. The one or more processors are used to analyze the one or more labeled digital images to generate a health index of the person.

Example 2 includes the method of example 1 wherein the one or more digital images comprise infrared (IR) images.

Example 3 includes the method of any of examples 1-2 wherein the one or more digital images comprise RGB (red, green, blue) images.

Example 4 includes the method of any of examples 1-3 wherein the one or more digital images are captured at a same time each day comprising a time proximate a waking or going to sleep time and the context information is collected contemporaneously with the capture of the one or more digital images.

Example 5 includes the method of any of examples 1-4 wherein collecting context information comprises collecting input by the person regarding how the person is feeling.

Example 6 includes the method of any of examples 1-5 wherein the individual health model comprises a convolutional neural network (CNN) trained with labeled images of the person, wherein the labels comprise medical conditions.

Example 7 includes the method of any of examples 1-6 and further including providing the labeled digital images and context information from the individual health model to a general health model, and receiving health condition information from the general health model responsive to the provided labeled digital images and context information, and using such received health condition information from the general health model in generating the health index.

Example 8 includes the method of any of examples 1-7 wherein the captured digital images are used to further train the individual health model.

Example 9 includes the method of any of examples 1-8 and further including providing the generated health index to a notification module, generating a notification including health advice for the person, and generating an appointment screen for a healthcare provider responsive to the generated health index being provided so the healthcare provider.

Example 10 includes the method of any of examples 1-9 wherein the camera is integrated into a cellular phone having a microbolometer array for capturing the digital images of the person representative of blood circulation.

In example 11, a device includes a memory storage comprising instructions, a camera, and one or more processors in communication with the memory storage and camera. The one or more processors execute the instructions to capture, via the camera, one or more digital images of a face of a person representative of blood circulation of the person, collect context information corresponding to the person contemporaneously with the capturing of the one or more digital images, label, via a trained individual health model executing on the one or more processors, the one or more digital images based on the blood circulation represented in the digital images and the collected context information via the trained individual heal model that has been trained on prior such digital images and context information, and analyze the one or more labeled digital images to generate a health index representative of the health of the person.

Example 12 includes the device of example 11 wherein the one or more digital images comprise infrared (IR) images.

Example 13 includes the device of any of examples 11-12 wherein the one or more digital images are captured at a same time each day comprising a time proximate a waking or going to sleep time and the context information is collected contemporaneously with the capture of the one or more digital images.

Example 14 includes the device of any of examples 11-13 wherein the individual health model comprises a convolutional neural network (CNN) trained with labeled images of the person, wherein the labels comprise medical conditions.

Example 15 includes the device of any of examples 11-14 wherein the one or more processors execute instructions to provide the labeled digital images and context information from the individual health model to a general health model, and receive health condition information from the general health model responsive to the provided labeled digital images and context information, and use such received health condition information from the general health model in generating the health index.

Example 16 includes the device of any of examples 11-15 wherein the one or more processors execute instructions to generate a notification including health advice for the person.

Example 17 includes the device of any of examples 11-16 wherein the device comprises a cellular phone with an integrated camera having a microbolometer array for capturing the digital images of the person representative of blood circulation.

In example 18, a non-transitory computer-readable media stores computer instructions for generating a health indication, that when such computer instructions are executed by one or more processors cause the one or more processors to perform operations including capturing, via a camera, one or more digital images of a face of a person representative of blood circulation of the person, collecting context information via one or more processors corresponding to the person contemporaneously with the capturing of the one or more digital images, labeling, via a trained individual health model executing on the one or more processors, the one or more digital images based on the blood circulation represented in the digital images and the collected context information via the trained individual heal model that has been trained on prior such digital images and context information, and analyzing, via the one or more processors, the one or more labeled digital images to generate a health index representative of the health of the person.

Example 19 includes the non-transitory computer-readable media of example 18 wherein the individual health model comprises a convolutional neural network (CNN) trained with labeled images of the person, wherein the labels comprise medical conditions, and wherein the labeled and captured images comprise infrared (IR) images.

Example 20 includes the non-transitory computer-readable media of any of examples 18-19 wherein executing the instructions further causes the one or more processors to perform operations including providing the labeled digital images and context information from the individual health model to a general health model, and receiving health condition information from the general health model responsive to the provided labeled digital images and context information, and using such received health condition information from the general health model in generating the health index.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

What is claimed is:

1. A computer implemented method comprising:
capturing, via a camera, one or more digital images of a face of a person representative of blood circulation of the person;
collecting context information via one or more processors corresponding to the person contemporaneously with the capturing of the one or more digital images;
labeling, via a trained individual health model executing on the one or more processors, the one or more digital images based on the blood circulation represented in the image and the collected context information via the trained individual health model that has been trained on prior such digital images and context information; and
analyzing, via the one or more processors, the one or more labeled digital images to generate a health index of the person.

2. The method of claim 1 wherein the one or more digital images comprise infrared (IR) images.

3. The method of claim 1 wherein the one or more digital images comprise RGB (red, green, blue) images.

4. The method of claim 1 wherein the one or more digital images are captured at a same time each day comprising a time proximate a waking or going to sleep time and the context information is collected contemporaneously with the capture of the one or more digital images.

5. The method of claim 1 wherein collecting context information comprises collecting input by the person regarding how the person is feeling.

6. The method of claim 1 wherein the individual health model comprises a convolutional neural network (CNN) trained with labeled images of the person, wherein the labels comprise medical conditions.

7. The method of claim 1 and further comprising:
providing the labeled digital images and context information from the individual health model to a general health model; and
receiving health condition information from the general health model responsive to the provided labeled digital images and context information, and using such received health condition information from the general health model in generating the health index.

8. The method of claim 1 wherein the captured digital images are used to further train the individual health model.

9. The method of claim 1 and further comprising:
providing the generated health index to a notification module;
generating a notification including health advice for the person; and
generating an appointment screen for a healthcare provider responsive to the generated health index being provided so the healthcare provider.

10. The method of claim 1 wherein the camera is integrated into a cellular phone having a microbolometer array for capturing the digital images of the person representative of blood circulation.

11. A device comprising:
a memory storage comprising instructions;
a camera; and
one or more processors in communication with the memory storage and camera, wherein the one or more processors execute the instructions to:
capture, via the camera, one or more digital images of a face of a person representative of blood circulation of the person;
collect context information corresponding to the person contemporaneously with the capturing of the one or more digital images;
label, via a trained individual health model executing on the one or more processors, the one or more digital images based on the blood circulation represented in the digital images and the collected context information via the trained individual heal model that has been trained on prior such digital images and context information; and
analyze the one or more labeled digital images to generate a health index representative of the health of the person.

12. The device of claim 11 wherein the one or more digital images comprise infrared (IR) images.

13. The device of claim 11 wherein the one or more digital images are captured at a same time each day comprising a time proximate a waking or going to sleep time and the context information is collected contemporaneously with the capture of the one or more digital images.

14. The device of claim 11 wherein the individual health model comprises a convolutional neural network (CNN) trained with labeled images of the person, wherein the labels comprise medical conditions.

15. The device of claim 11 wherein the one or more processors execute instructions to:
provide the labeled digital images and context information from the individual health model to a general health model; and
receive health condition information from the general health model responsive to the provided labeled digital images and context information, and use such received health condition information from the general health model in generating the health index.

16. The device of claim 11 wherein the one or more processors execute instructions to generate a notification including health advice for the person.

17. The device of claim 11 wherein the device comprises a cellular phone with an integrated camera having a microbolometer array for capturing the digital images of the person representative of blood circulation.

18. A non-transitory computer-readable media storing computer instructions for generating a health indication, that when such computer instructions are executed by one or more processors cause the one or more processors to perform operations comprising:
capturing, via a camera, one or more digital images of a face of a person representative of blood circulation of the person;
collecting context information via one or more processors corresponding to the person contemporaneously with the capturing of the one or more digital images;
labeling, via a trained individual health model executing on the one or more processors, the one or more digital images based on the blood circulation represented in the digital images and the collected context information via the trained individual heal model that has been trained on prior such digital images and context information; and
analyzing, via the one or more processors, the one or more labeled digital images to generate a health index representative of the health of the person.

19. The non-transitory computer-readable media of claim 18 wherein the individual health model comprises a convolutional neural network (CNN) trained with labeled images of the person, wherein the labels comprise medical conditions, and wherein the labeled and captured images comprise infrared (IR) images.

20. The non-transitory computer-readable media of claim 18 wherein executing the instructions further causes the one or more processors to perform operations comprising:
providing the labeled digital images and context information from the individual health model to a general health model; and
receiving health condition information from the general health model responsive to the provided labeled digital images and context information, and using such received health condition information from the general health model in generating the health index.
